(19) United States
(12) Patent Application Publication
(10) Pub. No.: US 2025/0042908 A1
(43) Pub. Date: Feb. 6, 2025

(54) SALTS FOR MTOR COMPOUNDS

(71) Applicant: GALDERMA HOLDING S.A., Zug (CH)

(72) Inventors: Craig Steven HARRIS, Biot (FR); Loïc Jean-Marc Frédéric TOMAS, La Tour de Peilz (CH)

(21) Appl. No.: 18/913,624

(22) Filed: Oct. 11, 2024

Related U.S. Application Data
(63) Continuation of application No. PCT/IB2023/053643, filed on Apr. 10, 2023.
(60) Provisional application No. 63/330,138, filed on Apr. 12, 2022.

(51) Int. Cl. C07D 487/04; A61K 31/519; C07C 55/08; C07C 59/245

(52) U.S. Cl. CPC C07D 487/04; A61K 31/519; C07C 55/08; C07C 59/245; C07B 2200/13

(57) ABSTRACT

The present technology provides the crystalline phosphate, malonate, and malate salts of 3-(2-amino-1,3-benzoxazol-5-yl)-1-(1,3-dimethylbutyl)pyrazolo[3,4-d]pyrimidine-4,6-diamine. The present technology further provides methods for preparing the various forms, compositions containing them, and methods related to modulation of mTOR. In particular, the present crystalline compounds and compositions may be used to treat mTOR-mediated disorders and conditions, including, e.g., dermatological disorders related to a disorder of keratinization with a proliferative, inflammatory and/or immuno-allergic component.

### RELATED APPLICATIONS

This application is a continuation of International Patent Application No. PCT/IB2023/053643 filed Apr. 10, 2023, which application claims priority under 35 U.S.C. § 119(e) to U.S. Provisional Application No. 63/330,138 filed Apr. 12, 2022, the entire contents of both of which are incorporated herein by reference.

### FIELD OF INVENTION

The present technology relates to generally to crystalline forms of 3-(2-amino-1,3-benzoxazol-5-yl)-1-(1,3-dimethylbutyl)pyrazolo[3,4-d]pyrimidine-4,6-diamine, its salts, methods for preparing them, compositions containing them, and methods of treatment employing them.

### BACKGROUND

3-(2-Amino-1,3-benzoxazol-5-yl)-1-(1,3-dimethylbutyl)pyrazolo[3,4-d]pyrimidine-4,6-diamine (described in PCT/EP2018/086066, filed Dec. 20, 2018, and incorporated by reference in its entirety herein) has the structure of compound of Formula I (Compound I):

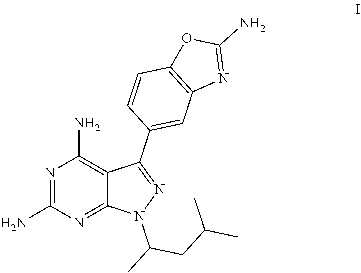 [SMILES: CC(C)CC(C)n1nc(-c2ccc3oc(N)nc3c2)c2c(N)nc(N)nc21]

Compound I is an inhibitor of mammalian target of rapamycin (mTOR) and is useful in the treatment of mTOR-mediated disorders and conditions, including, e.g., dermatological disorders related to a disorder of keratinization with a proliferative, inflammatory and/or immuno-allergic component such as psoriasis, atopic dermatitis, actinic keratosis, or acne.

It is well known that the crystalline form of the active pharmaceutical ingredient (API) of a particular drug is often an important determinant of the drug's ease of preparation, hygroscopicity, stability, solubility, storage stability, ease of formulation, rate of dissolution in gastrointestinal fluids and in vivo bioavailability. Crystalline forms occur where the same composition of matter crystallizes in a different lattice arrangement resulting in different thermodynamic properties and stabilities specific to the particular crystalline form. Crystalline forms may also include different hydrates or solvates of the same compound. In deciding which form is preferable, the numerous properties of the forms are compared and the preferred form chosen based on the many physical property variables. It is entirely possible that one form can be preferable in some circumstances where certain aspects such as ease of preparation, stability, etc. are deemed to be critical. In other situations, a different form may be preferred for greater dissolution rate and/or superior bioavailability. It is not yet possible to predict whether a particular compound or salt of a compound will form polymorphs (i.e., crystal structures), whether any such polymorphs will be suitable for commercial use in a therapeutic composition, or which polymorphs will display such desirable properties.

### SUMMARY

Provided herein are salts of Compound I, wherein the salt is selected from phosphate, malonate, and malate. Further provided herein are crystal forms of Compound I, compositions including the crystal forms, and methods of preparing the crystal forms and compositions. The present technology further provides methods of using the crystal forms of Compound I and compositions thereof to treat mTOR-mediated disorders or conditions, including, but not limited to psoriasis, atopic dermatitis, actinic keratosis, or acne.

### BRIEF DESCRIPTION OF THE DRAWINGS

FIG. 1 provides an XRPD spectrum of the crystalline phosphate salt of Compound I, according to the examples. The XRPD pattern recorded for the phosphate salt (bottom trace) is similar to the XRPD pattern recorded for a previously analyzed phosphate salt (top trace).

FIG. 2 provides a DSC (bottom trace) and TGA (top trace) thermogram of the crystalline phosphate salt of Compound I, according to the examples.

FIG. 3 provides an XRPD spectrum of the crystalline malonate salt of Compound I, according to the examples. The XRPD pattern recorded for the malonate salt (bottom trace) is different than the XRPD pattern recorded for a previously analyzed malonate salt (top trace). The middle trace is the XRPD pattern for malonic acid.

FIG. 4 provides a DSC (left trace) and TGA (right trace) thermogram of the crystalline malonate salt of Compound I, according to the examples.

FIG. 5 provides an FTIR spectrum of two batches of the crystalline malonate salt of Compound I, according to the examples.

FIG. 6 provides an XRPD spectrum of the crystalline malate salt of Compound I, according to the examples. The XRPD pattern recorded for the malate salt (bottom trace) is similar to the XRPD pattern recorded for a previously analyzed malate salt (top trace). The middle trace is the XRPD pattern for DL-malic acid.

FIG. 7 provides a DSC (bottom trace) and TGA (top trace) thermogram of the crystalline malate salt of Compound I, according to the examples.

FIG. 8 provides a comparison of the XRPD pattern recorded for solid residues collected on water solubility assay done for Compound I free base compared to XRPD pattern recorded for Compound I as received.

FIG. 9 provides a comparison of XRPD pattern recorded for solid residues collected on water solubility assay done for Compound I phosphate salt compared to XRPD pattern recorded for Compound I phosphate salt as received.

FIG. 10 provides a comparison of XRPD pattern recorded for solid residues collected on water solubility assay done for Compound I malonate salt compared to XRPD pattern recorded for Compound I malonate salt as received.

FIG. 11 provides a comparison XRPD pattern recorded for solid residues collected on water solubility assay done for Compound I malate salt compared to XRPD pattern recorded for Compound I malate salt as received.

### DETAILED DESCRIPTION

In one aspect, the present technology provides salts of compound of Formula I, wherein the salt is selected from phosphate, malonate, and malate. In some embodiments, the salt is crystalline.

In another aspect, the present technology provides a crystalline compound selected from a crystalline phosphate, malonate, or malate salt of the compound of Formula I:

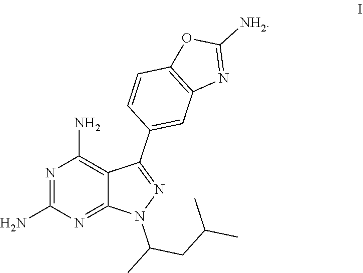 [SMILES: CC(C)CC(C)n1nc(-c2ccc3oc(N)nc3c2)c2c(N)nc(N)nc21]

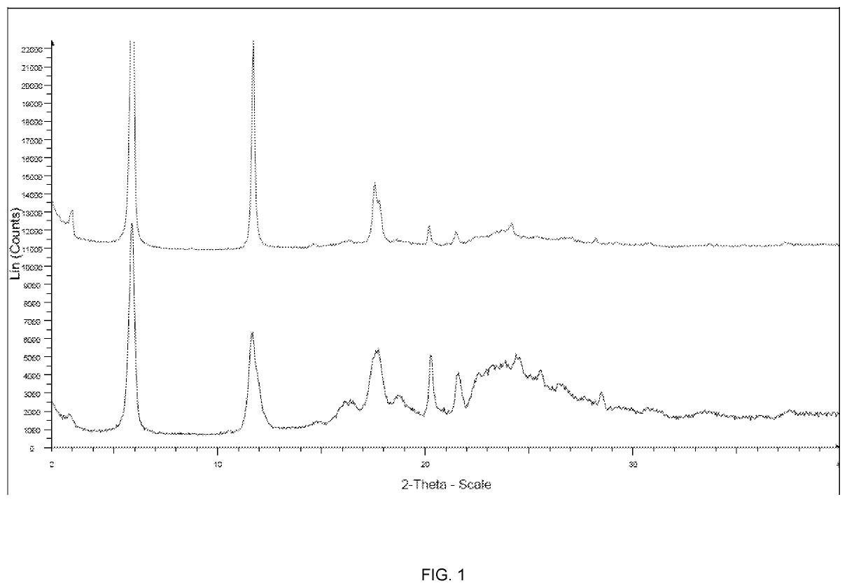

In one embodiment, the present technology provides the crystalline phosphate salt of Compound I. In any embodiment, the crystalline phosphate salt may have an XRPD including a characteristic peak, in terms of 2θ, at about 5.8°. In any embodiment, the crystalline phosphate salt may have an XRPD including one or more characteristic peaks, in terms of 2θ, at about 5.8°, about 11.6°, and about 17.7°. In any embodiment, the crystalline phosphate salt may further include one or more characteristic peaks, in terms of 2θ, at about 24.4°, about 20.3°, and about 23.8°. In any embodiment, the crystalline phosphate salt may further include have one or more characteristic peaks, in terms of 2θ, at about 23.2°, about 21.6°, about 22.6°, and about 26.6°. In any embodiment, the crystalline phosphate salt may have an XRPD substantially as shown in FIG. 1.

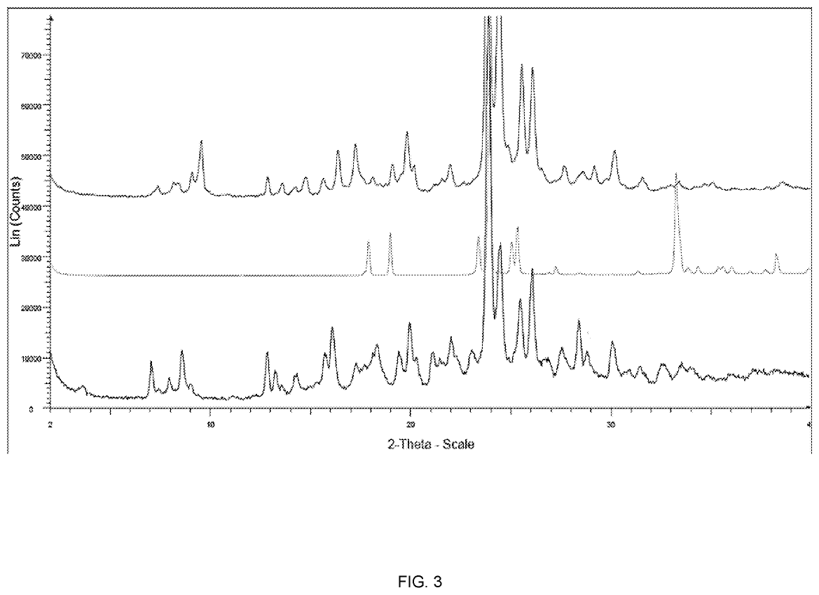

In one embodiment, the present technology provides the crystalline malonate salt of Compound I. In any embodiment, the malonate salt may have an XRPD including a characteristic peak, in terms of 2θ, at about 24.0°. In any embodiment, the crystalline malonate salt may have an XRPD including one or more characteristic peaks, in terms of 2θ, at about 24.0°, about 24.6°, and about 26.2°. In any embodiment, the crystalline malonate salt may further include one or more characteristic peaks, in terms of 2θ, at about 25.6°, about 20.1°, and about 16.2°. In any embodiment, the crystalline malonate salt may further include one or more characteristic peaks, in terms of 2θ, at about 22.2°, about 30.2°, about 18.5°, about 8.7°, and about 23.2°. In any embodiment, the crystalline malonate salt may have an XRPD substantially as shown in FIG. 3.

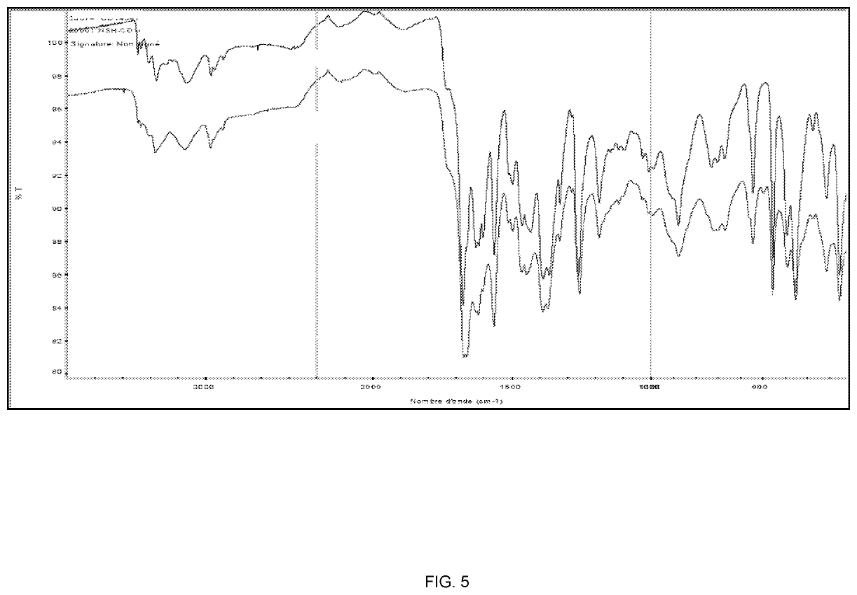

In one embodiment, the present technology provides the crystalline malate salt of Compound I. In any embodiment, the crystalline malate salt may have an XRPD including a characteristic peak, in terms of 2θ, at about 5.7°. In any embodiment, the crystalline malate salt may have an XRPD including one or more characteristic peaks, in terms of 2θ, at about 5.7°, about 21.3°, and about 11.4°. In any embodiment, the crystalline malate salt may further include one or more characteristic peaks, in terms of 2θ, at about 21.9°, about 23.2°, and about 14.4°, and about 8.9°. In any embodiment, the crystalline malate salt may have an XRPD substantially as shown in FIG. 5.

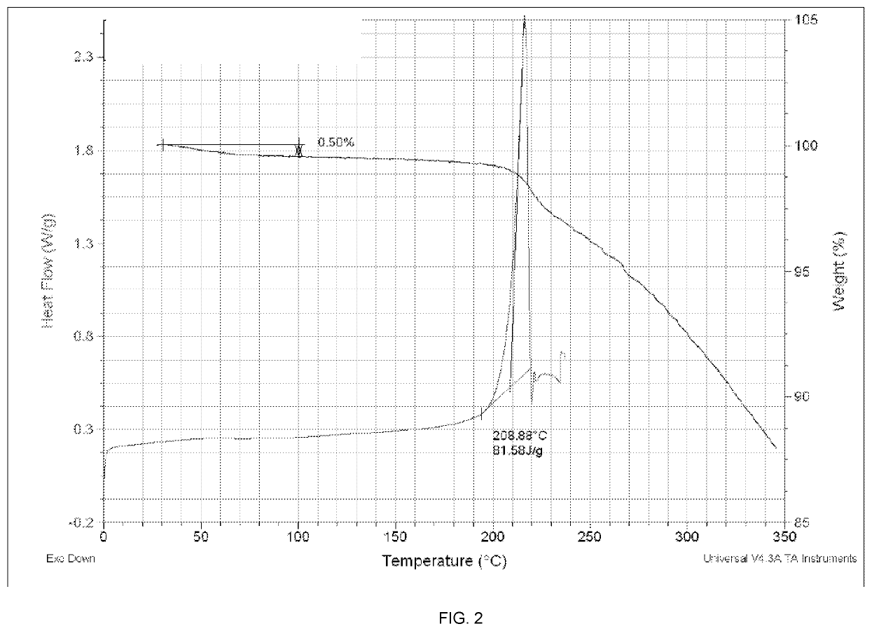

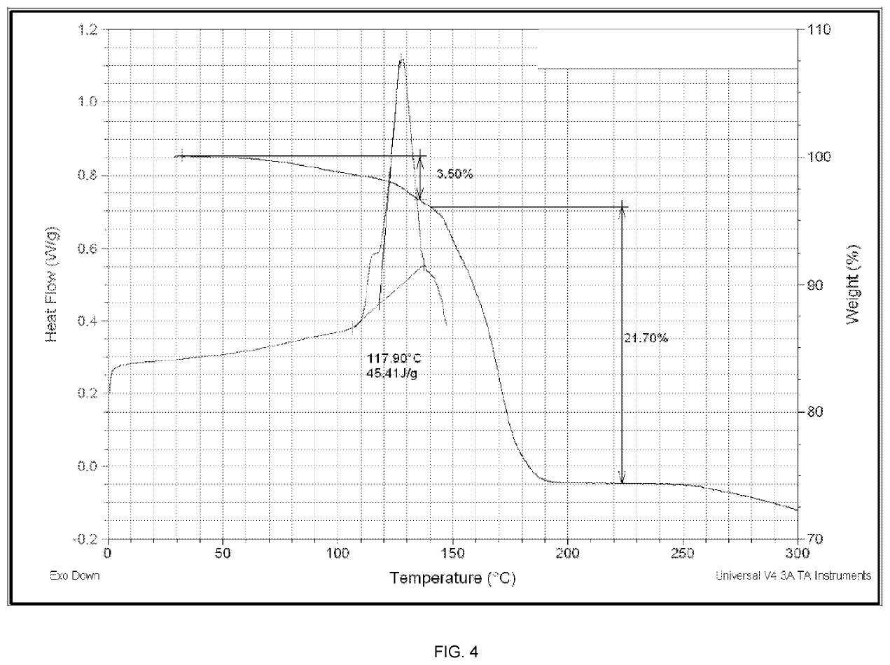

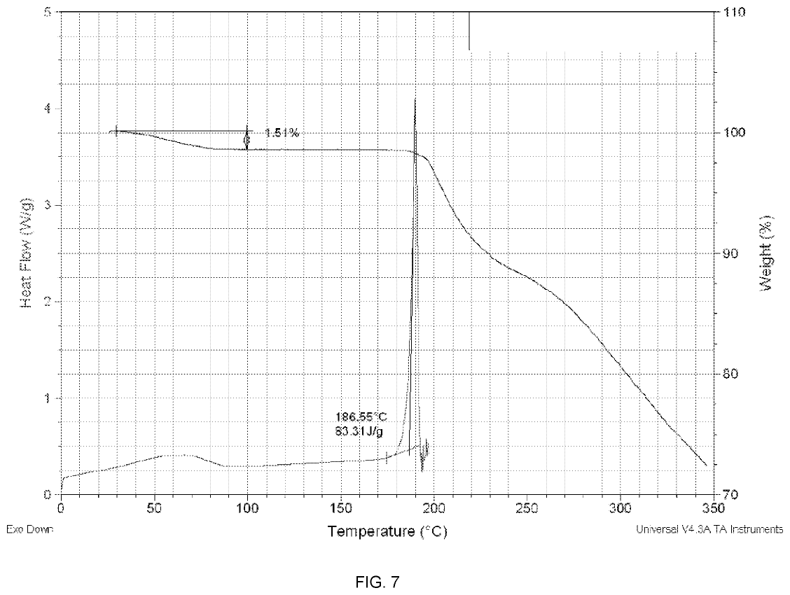

The crystalline compounds may be characterized thermally. In any embodiment, the crystalline phosphate salt may have a DSC thermogram showing an onset of an endotherm at about 208.9° C. In any embodiment, the crystalline phosphate salt may have a DSC thermogram substantially as shown in FIG. 2. In any embodiment, the crystalline malonate salt may have a DSC thermogram showing an onset of an endotherm at about 117.9° C. In any embodiment, the crystalline malonate salt may have a DSC thermogram substantially as shown in FIG. 4. In any embodiment, the crystalline malate salt may have a DSC thermogram showing an onset of an endotherm at about 186.6° C. In any embodiment, the crystalline malate salt may have a DSC thermogram substantially as shown in FIG. 7.

The crystalline compounds may be characterized thermally. In any embodiment, the crystalline phosphate salt may have a TGA thermogram demonstrating a weight loss of about 0.5% (up to about 100° C.). In any embodiment, the crystalline phosphate salt may have a TGA thermogram substantially as shown in FIG. 2. In any embodiment, the crystalline malonate salt may have a TGA thermogram demonstrating a weight loss of about 3.5% (up to about 140° C.). In any embodiment, the crystalline malonate salt may have a TGA thermogram substantially as shown in FIG. 4. In any embodiment, the crystalline malate salt may have a TGA thermogram demonstrating a weight loss of about 1.5% (up to about 100° C.). In any embodiment, the crystalline malate salt may have a TGA thermogram substantially as shown in FIG. 7.

In any embodiment, the crystalline compound of Compound I may have an XRPD including one, two, three, four, five, six, seven, eight, nine, or ten characteristic peaks, in terms of 20.

In some embodiments, the crystalline compound of Compound I may be anhydrous. In any embodiment, the crystalline phosphate salt of Compound I may be anhydrous. In any embodiment, the crystalline malonate salt of Compound I may be anhydrous. In any embodiment, the crystalline malate salt of Compound I may be anhydrous.

In some embodiments, the crystalline compound of Compound I may be a hydrate. In any embodiment, the crystalline phosphate salt of Compound I may be a hydrate (e.g., a monohydrate). In any embodiment, the crystalline malonate salt of Compound I may be a hydrate (e.g., a monohydrate). In any embodiment, the crystalline malate salt of Compound I may be a hydrate (e.g., a monohydrate).

As used herein, with respect to values of 20, the terms “about” and “substantially” indicate that such values for individual peaks can vary by ±0.4°. In some embodiments, the values of 20 for individual peaks can vary by ±0.3°. In some embodiments, the values of 20 for individual peaks can vary by ±0.2°.

As used herein, with respect to features such as endotherms, exotherms, baseline shifts, etc., the terms “about” and “substantially” indicate that their values can vary+4° C. For DSC, variation in the temperatures observed will depend upon the rate of temperature change as well as sample preparation technique and the particular instrument employed. Thus, the values reported herein relating to DSC thermograms can vary ±4° C. For TGA, variation in the temperatures observed will depend upon the rate of temperature change as well as sample preparation technique and the particular instrument employed. Thus, the values reported herein relating to TGA thermograms can vary ±4° C.

As used herein, other than with respect to values of 20, endotherms, exotherms, and baseline shifts as defined above, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

In addition to the techniques described above for characterizing crystalline forms of as described herein, XRPD, single crystal X-ray diffraction, DSC, dynamic vapor sorption (DVS), crystal morphology, solid state nuclear magnetic resonance, Raman scattering, infrared (IR) spectroscopy, and/or polarized light microscopy (PLM) may also be useful for characterization of other crystalline or amorphous forms of the present technology.

In another aspect, the present technology provides a method of making the crystalline compounds of Compound I disclosed herein.

In another embodiment, the crystalline free base of Compound I (form A) may be provided by contacting amorphous Compound I with an organic solvent. In any embodiment, the organic solvent may include acetonitrile. In any embodiment, the organic solvent may include acetonitrile and one or more other organic solvents. In any embodiment, the method may further include filtering the precipitate. In any embodiment, the method may further include drying the precipitate. In any embodiment, the contacting occurs at room temperature (i.e., between about 20° C. to about 25° C.). In any embodiment, the contacting occurs for about 1 h to about 7 days, about 3 days to about 7 days, about 2 hours to about 14 hours, about 2 h to about 10 h, or about 2 hours to about 5 hours.

In any embodiment, the crystalline phosphate salt of Compound I may be provided by contacting a suspension of free base in one or more organic solvents with phosphoric acid (“H₃PO₄”) to precipitate the crystalline phosphate salt. In any embodiment, the organic solvent may include ethanol. In any embodiment, the suspension may turn into a homogenous solution. In any embodiment, the method may include heating the suspension or homogenous solution to a temperature less than or equal to about 60° C. In any embodiment, the heating may occur for at least 1 h. In any embodiment, the method may include cooling the solution or homogenous solution. In any embodiment, the method may further include filtering the precipitate. In any embodiment, the method may further include drying the precipitate.

In any embodiment, the crystalline malonate salt of Compound I may be provided by contacting a suspension of the free base with malonic acid to precipitate the crystalline malonate salt. In any embodiment, the contacting takes place in the presence of one or more organic solvents. In any embodiment, the organic solvent may include methyl ethyl ketone. In any embodiment, the organic solvent is methyl ethyl ketone. In any embodiment, the suspension may turn into a homogenous solution. In any embodiment, the method may include heating the suspension or homogenous solution to a temperature less than or equal to about 60° C. In any embodiment, the heating may occur for at least 1 h. In any embodiment, the method may include cooling the solution or homogenous solution. In any embodiment, the method may further include filtering the precipitate. In any embodiment, the method may further include drying the precipitate.

In any embodiment, the crystalline malate salt of Compound I may be provided by contacting a suspension of free base in one or more organic solvents with (S)-Malic acid to precipitate the crystalline malate salt. In any embodiment, the organic solvent may include acetonitrile. In any embodiment, the suspension may turn into a homogenous solution. In any embodiment, the method may include heating the suspension or homogenous solution to a temperature less than or equal to about 60° C. In any embodiment, the heating may occur for at least 1 h. In any embodiment, the method may include cooling the solution or homogenous solution. In any embodiment, the method may further include filtering the precipitate. In any embodiment, the method may further include drying the precipitate.

In any embodiment, the crystals may be further subjected to steps such as, e.g., drying, purification, etc. In any embodiment, the crystals may be filtered. In any embodiment, the crystals may be subjected to drying at a suitable temperature. In one embodiment, the crystals may be dried at a temperature in the range of about 200° C. to about 60° C. In some embodiments, the crystals may be dried at a temperature in the range of about 20° C. to about 250° C. In some embodiments, the crystals may be dried at a temperature in the range of about 350° C. to about 550° C. In some embodiments, the crystals may be dried under reduced pressure in the range, for example, of about 10 mbar-about 40 mbar. The drying step may be conducted for a suitable period of time. Thus in one embodiment, the crystals are dried for a period of about 1 to about 72 hours, from about 2 to about 36 hours or from about 4 to about 18 hours. In some embodiments, the crystals are dried for about 48 h.

Crystalline forms as described herein may be isolated in substantially pure form. By “substantially pure” it is meant that more than 50% by weight of Compound I is present in one of the crystalline forms disclosed herein. In some embodiments of the isolated or substantially pure crystalline forms as described herein, Compound I may be present at least about 60%, at least about 70%, at least about 80%, at least about 90%, at least about 95%, at least about 97%, at least about 98%, or at least about 99% by weight of the indicated form. For example, in certain embodiments, the present technology provides crystals wherein at least about 50%, at least about 70%, at least about 80%, at least about 90%, at least about 95%, at least about 97%, at least about 98%, or at least about 99% by weight may be the crystal present.

The present technology also provides a pharmaceutical composition, which includes an effective amount of one or more of the crystalline forms as described herein for treating an mTOR-mediated disorder or condition. The mTOR-mediated disorder or condition may be a dermatological disorders related to a disorder of keratinization with a proliferative, inflammatory and/or immuno-allergic component such as psoriasis, atopic dermatitis, actinic keratosis, or acne.

In a further related aspect, a method is provided that includes administering an effective amount of one or more of the crystalline forms as described herein or administering a pharmaceutical composition comprising an effective amount of one or more of the crystalline forms as described herein to a subject suffering from an mTOR-mediated disorder or condition. The mTOR-mediated disorder or condition may be dermatological disorders related to a disorder of keratinization with a proliferative, inflammatory and/or immuno-allergic component. In some embodiments, the dermatological disorder may be psoriasis, atopic dermatitis, actinic keratosis, or acne.

In a further related aspect, a method is provided that includes modulating mTOR by contacting mTOR with an effective amount of one or more of the crystalline forms as described herein.

“Effective amount” refers to the amount of a crystalline form as described herein or compositions thereof as described herein required to produce a desired effect. One example of an effective amount includes amounts or dosages that yield acceptable toxicity and bioavailability levels for therapeutic (pharmaceutical) use including, but not limited to, a dermatological complaint associated with a keratinization disorder with a proliferative, inflammatory and/or immunoallergic component. Another example of an effective amount includes amounts or dosages that are capable of reducing symptoms associated with psoriasis, atopic dermatitis, actinic keratosis or acne. The effective amount of the crystalline form as described herein may selectively modulate mTOR. As used herein, a “subject” or “patient” is a mammal, such as a cat, dog, rodent or primate. Typically, the subject is a human, and, preferably, a human suffering from or suspected of suffering from an mTOR-mediated disorder or condition. The term “subject” and “patient” can be used interchangeably.

In still another aspect, the present technology provides methods of modulating mTOR by contacting mTOR with an effective amount of one or more of the crystalline forms as described herein.

Thus, the instant present technology provides pharmaceutical compositions and medicaments comprising any of the crystalline forms disclosed herein and a pharmaceutically acceptable carrier or one or more excipients or fillers. The compositions may be used in the methods and treatments described herein. Such compositions and medicaments include a therapeutically effective amount of one or more of the crystalline forms as described herein. The pharmaceutical composition may be packaged in unit dosage form.

The pharmaceutical compositions and medicaments may be prepared by mixing one or more the crystalline forms disclosed herein with pharmaceutically acceptable carriers, excipients, binders, diluents or the like to prevent and treat disorders associated with the effects of increased plasma and/or hepatic lipid levels. The one or more crystalline forms disclosed herein and compositions thereof as described herein may be used to prepare formulations and medicaments that prevent or treat a variety of disorders associated with or mediated by mTOR, including but not limited to a dermatological complaint associated with a keratinization disorder with a proliferative, inflammatory and/or immunoallergic component, for example psoriasis, atopic dermatitis, actinic keratosis or acne. Such compositions can be in the form of, for example, granules, powders, tablets, capsules, syrup, suppositories, injections, emulsions, elixirs, suspensions or solutions. The instant compositions can be formulated for various routes of administration, for example, by oral, parenteral, topical, rectal, nasal, vaginal administration, or via implanted reservoir. Parenteral or systemic administration includes, but is not limited to, subcutaneous, intravenous, intraperitoneal, and intramuscular, injections. The following dosage forms are given by way of example and should not be construed as limiting the instant present technology.

For oral, buccal, and sublingual administration, powders, suspensions, granules, tablets, pills, capsules, gelcaps, and caplets are acceptable as solid dosage forms. These can be prepared, for example, by mixing one or more crystalline forms disclosed herein with at least one additive such as a starch or other additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms can contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, binders, thickeners, buffers, sweeteners, flavoring agents or perfuming agents. Tablets and pills may be further treated with suitable coating materials known in the art.

Liquid dosage forms for oral administration may be in the form of pharmaceutically acceptable emulsions, syrups, elixirs, suspensions, and solutions, which may contain an inactive diluent, such as water. Pharmaceutical formulations and medicaments may be prepared as liquid suspensions or solutions using a sterile liquid, such as, but not limited to, an oil, water, an alcohol, and combinations of these. Pharmaceutically suitable surfactants, suspending agents, emulsifying agents, may be added for oral or parenteral administration.

As noted above, suspensions may include oils. Such oils include, but are not limited to, peanut oil, sesame oil, cottonseed oil, corn oil and olive oil. Suspension preparation may also contain esters of fatty acids such as ethyl oleate, isopropyl myristate, fatty acid glycerides and acetylated fatty acid glycerides. Suspension formulations may include alcohols, such as, but not limited to, ethanol, isopropyl alcohol, hexadecyl alcohol, glycerol and propylene glycol. Ethers, such as but not limited to, poly(ethyleneglycol), petroleum hydrocarbons such as mineral oil and petrolatum; and water may also be used in suspension formulations.

Injectable dosage forms generally include aqueous suspensions or oil suspensions, which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. Alternatively, sterile oils may be employed as solvents or suspending agents. Typically, the oil or fatty acid is non-volatile, including natural or synthetic oils, fatty acids, mono-, di- or tri-glycerides.

For injection, the pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

Crystalline forms of the present technology may be administered to the lungs by inhalation through the nose or mouth. Suitable pharmaceutical formulations for inhalation include solutions, sprays, dry powders, or aerosols containing any appropriate solvents and optionally other compounds such as, but not limited to, stabilizers, antimicrobial agents, antioxidants, pH modifiers, surfactants, bioavailability modifiers and combinations of these. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aqueous and nonaqueous (e.g., in a fluorocarbon propellant) aerosols are typically used for delivery of crystalline forms of the present technology by inhalation.

Dosage forms for the topical (including buccal and sublingual) or transdermal administration of one or more crystalline forms disclosed herein include powders, sprays, ointments, pastes, creams, lotions, gels, solutions, and patches. The active component may be mixed under sterile conditions with a pharmaceutically-acceptable carrier or excipient, and with any preservatives, or buffers, which may be required. Powders and sprays can be prepared, for example, with excipients such as lactose, talc, silicic acid, aluminum hydroxide, calcium silicates and polyamide powder, or mixtures of these substances. The ointments, pastes, creams and gels may also contain excipients such as animal and vegetable fats, oils, waxes, paraffins, starch, tragacanth, cellulose derivatives, polyethylene glycols, silicones, bentonites, silicic acid, talc and zinc oxide, or mixtures thereof. Absorption enhancers can also be used to increase the flux of the one or more crystalline forms disclosed herein across the skin. The rate of such flux can be controlled by either providing a rate controlling membrane (e.g., as part of a transdermal patch) or dispersing the one or more crystalline forms disclosed herein in a polymer matrix or gel.

Besides those representative dosage forms described above, pharmaceutically acceptable excipients and carriers are generally known to those skilled in the art and are thus included in the instant present technology. Such excipients and carriers are described, for example, in “Remingtons Pharmaceutical Sciences” Mack Pub. Co., New Jersey (1991), which is incorporated herein by reference.

The formulations of the present technology may be designed to be short-acting, fast-releasing, long-acting, and sustained-releasing as described below. Thus, the pharmaceutical formulations may also be formulated for controlled release or for slow release.

The instant compositions may also comprise, for example, micelles or liposomes, or some other encapsulated form, or may be administered in an extended release form to provide a prolonged storage and/or delivery effect. Therefore, the pharmaceutical formulations and medicaments may be compressed into pellets or cylinders and implanted intramuscularly or subcutaneously as depot injections or as implants such as stents. Such implants may employ known inert materials such as silicones and biodegradable polymers.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant present technology.

Those skilled in the art are readily able to determine an effective amount by simply administering one or more crystalline forms disclosed herein to a patient in increasing amounts until for example, (for metabolic syndrome and/or obesity) the elevated plasma or elevated white blood cell count or hepatic cholesterol or triglycerides or progression of the disease state is reduced or stopped. For metabolic syndrome and/or obesity, the progression of the disease state can be assessed using in vivo imaging, as described, or by taking a tissue sample from a patient and observing the target of interest therein.

The one or more crystalline forms disclosed herein can be administered to a patient at dosage levels in the range of about 0.1 to about 1,000 mg per day. For a normal human adult having a body weight of about 70 kg, a dosage in the range of about 0.01 to about 100 mg per kg of body weight per day is sufficient. The specific dosage used, however, can vary or may be adjusted as considered appropriate by those of ordinary skill in the art. For example, the dosage can depend on a number of factors including the requirements of the patient, the severity of the condition being treated and the pharmacological activity of the compound being used. The determination of optimum dosages for a particular patient is well known to those skilled in the art.

Various assays and model systems can be readily employed to determine the therapeutic effectiveness of the treatment according to the present technology.

Effectiveness of the compositions and methods of the present technology may also be demonstrated by a decrease in the signs and symptoms of psoriasis, atopic dermatitis, actinic keratosis or acne.

For each of the indicated conditions described herein, test subjects will exhibit a 10%, 20%, 30%, 50% or greater reduction, up to a 75-90%, or 95% or greater, reduction, in one or more symptom(s) caused by, or associated with, the disorder in the subject, compared to placebo-treated or other suitable control subjects.

The one or more crystalline forms disclosed herein can also be administered to a patient along with other conventional therapeutic agents that may be useful in the treatment of a keratinization disorder with a proliferative, inflammatory and/or immunoallergic component, including psoriasis, atopic dermatitis, actinic keratosis or acne. The administration may include oral administration, parenteral administration, or nasal administration. In any of these embodiments, the administration may include subcutaneous injections, intravenous injections, intraperitoneal injections, or intramuscular injections. In any of these embodiments, the administration may include oral administration. The methods of the present technology can also comprise administering, either sequentially or in combination with one or more compounds of the present technology, a conventional therapeutic agent in an amount that can potentially be effective for the treatment of a keratinization disorder with a proliferative, inflammatory and/or immunoallergic component, including psoriasis, atopic dermatitis, actinic keratosis or acne.

In one aspect, one or more crystalline forms disclosed herein may be administered to a patient in an amount or dosage suitable for therapeutic use. Generally, a unit dosage comprising one or more the crystalline forms of the present technology will vary depending on patient considerations. Such considerations include, for example, age, protocol, condition, sex, extent of disease, contraindications, concomitant therapies and the like. An exemplary unit dosage based on these considerations can also be adjusted or modified by a physician skilled in the art. For example, a unit dosage for a patient comprising a compound of the present technology can vary from 1×10⁻⁴ g/kg to 1 g/kg, preferably, 1×10⁻³ g/kg to 1.0 g/kg. Dosage of a compound of the present technology can also vary from 0.01 mg/kg to 100 mg/kg or, preferably, from 0.1 mg/kg to 10 mg/kg.

A crystalline form of the present technology can also be modified, for example, by the covalent attachment of an organic moiety or conjugate to improve pharmacokinetic properties, toxicity or bioavailability (e.g., increased in vivo half-life). The conjugate can be a linear or branched hydrophilic polymeric group, fatty acid group or fatty acid ester group. A polymeric group can comprise a molecular weight that can be adjusted by one of ordinary skill in the art to improve, for example, pharmacokinetic properties, toxicity or bioavailability. Exemplary conjugates can include a polyalkane glycol (e.g., polyethylene glycol (PEG), polypropylene glycol (PPG)), carbohydrate polymer, amino acid polymer or polyvinyl pyrolidone and a fatty acid or fatty acid ester group, each of which can independently comprise from about eight to about seventy carbon atoms. Conjugates for use with a compound of the present technology can also serve as linkers to, for example, any suitable substituents or groups, radiolabels (marker or tags), halogens, proteins, enzymes, polypeptides, other therapeutic agents (for example, a pharmaceutical or drug), nucleosides, dyes, oligonucleotides, lipids, phospholipids and/or liposomes. In one aspect, conjugates can include polyethylene amine (PEI), polyglycine, hybrids of PEI and polyglycine, polyethylene glycol (PEG) or methoxypolyethylene glycol (mPEG). A conjugate can also link a compound of the present technology to, for example, a label (fluorescent or luminescent) or marker (radionuclide, radioisotope and/or isotope) to comprise a probe of the present technology. Conjugates for use with a compound of the present technology can, in one aspect, improve in vivo half-life. Other exemplary conjugates for use with a compound of the present technology as well as applications thereof and related techniques include those generally described by U.S. Pat. No. 5,672,662, which is hereby incorporated by reference herein.

In another aspect, the present technology provides methods of identifying a target of interest including contacting the target of interest with a detectable or imaging effective quantity of a labeled crystalline form of the present technology. A detectable or imaging effective quantity is a quantity of a labeled crystal of the present technology necessary to be detected by the detection method chosen. For example, a detectable quantity can be an administered amount sufficient to enable detection of binding of the labeled compound to a target of interest including, but not limited to, a KOR. Suitable labels are known by those skilled in the art and can include, for example, radioisotopes, radionuclides, isotopes, fluorescent groups, biotin (in conjunction with streptavidin complexation), and chemoluminescent groups. Upon binding of the labeled crystal to the target of interest, the target may be isolated, purified and further characterized such as by determining the amino acid sequence.

The terms “associated” and/or “binding” can mean a chemical or physical interaction, for example, between a compound of the present technology and a target of interest. Examples of associations or interactions include covalent bonds, ionic bonds, hydrophilic-hydrophilic interactions, hydrophobic-hydrophobic interactions and complexes. Associated can also refer generally to “binding” or “affinity” as each can be used to describe various chemical or physical interactions. Measuring binding or affinity is also routine to those skilled in the art. For example, crystalline forms of the present technology can bind to or interact with a target of interest or precursors, portions, fragments and peptides thereof and/or their deposits.

Treatment within the context of the instant technology, means an alleviation, in whole or in part, of symptoms associated with a disorder or disease, or slowing or halting of further progression or worsening of those symptoms, or tending to prevent or ward off the disease or disorder in a subject at risk for developing the disease or disorder.

The examples herein are provided to illustrate advantages of the present technology and to further assist a person of ordinary skill in the art with preparing or using the compounds of the present technology or salts, pharmaceutical compositions, derivatives, solvates, metabolites, prodrugs, racemic mixtures or tautomeric forms thereof. The examples herein are also presented in order to more fully illustrate the preferred aspects of the present technology. The examples should in no way be construed as limiting the scope of the present technology, as defined by the appended claims. The examples can include or incorporate any of the variations, aspects or aspects of the present technology described above. The variations, aspects or aspects described above may also further each include or incorporate the variations of any or all other variations, aspects or aspects of the present technology.

The present technology, thus generally described, will be understood more readily by reference to the following examples, which are provided by way of illustration and are not intended to be limiting of the present technology.

### EXAMPLES

The following abbreviations are used throughout the present disclosure with respect to chemical and biological terminology:
DMSO: Dimethylsulfoxide
DSC: Differential scanning calorimetry
FT-IR: Fourier transform-infrared
HPLC: High performance liquid chromatography
NMR: Nuclear magnetic resonance spectroscopy
RT: Room temperature
TGA: Thermo gravimetric analysis
UPLC-UV: Ultra Performance Liquid Chromatograph-Ultraviolet
UV: Ultraviolet
XRPD: X-ray powder diffraction

### General Methods, Instruments, and Standards Employed

### X-Ray Powder Diffraction (XRPD)

XRPD analysis was conducted with a Bruker D8-Advance diffractometer, type: Bragg-Bentano. Source CuKα1, λ=1.5406 Å and CuKα2, λ2=1.54439 Å. Generator: 35 kV-40 mA. Detector: Lynx Eye.

The sample was analyzed using the following protocol: Around 3 mg of the test compound delivered as solid material is gently grounded to homogenize the powder and deposed on zero-diffraction Silicon sample holder. The sample holder is then placed in the X-Ray Diffractometer chamber.

The copper anode X-ray source is turned on using a generator set at 35 kV voltage and at 40 mA intensity. While X-ray beam irradiates the sample, both X-ray source and X-ray detector move on the goniometer with equal 0 angle: diffractometer in Bragg-Brentano configuration. XRPD pattern is recorded in the [2°; 40°] 2θ angle range by step of 0.040 in 20 and for 1 second at each angle step.

### Differential Scanning Calorimetry (DSC)

DSC was performed using a TA Q200 DSC from TA Instruments. Standard pan: TA 901670-901 not hermetic. Standard lid: TA 901671-901.

The sample were analyzed as follows: Around 2 mg of the Test compound delivered as solid material is accurately weighed in an aluminum pan. The pan, cover with a special lid pan, is then crimped. The sample pan and a reference empty pan are placed in the instrument cell to start DSC measurements. An heating run is then applied to the test compound at a constant heating rate of 10° C./min from 0° C. to 350° C. to record the DSC thermogram of the test compound. The Thermal behavior of the test compound is reported as a DSC thermogram reporting the recorded heat flux as function of temperature.

### Thermogravimetric Analysis (TGA)

TGA data was collected using a TA Q500 TGA from TA Instruments. Standard pan: TA 901670-901 not hermetic. Standard lid: TA 901671-901.

Analysis was conducted as follows: Around 5-10 mg of the Test compound delivered as solid material is placed in an aluminum pan. The sample pan, cover with a special lid pan, is supported by a precision balance and placed in the instrument furnace. An heating program is then applied to the test compound at a constant heating rate of 10° C./min from RT to 350° C. to record the TGA thermogram of the test compound. Specific temperature programs involving isothermal and different heating steps can be applied on the sample.

The TGA thermogram of the test compound reports the recorded sample mass or the calculated % of the sample loss of mass as function of temperature.

### Proton Nuclear Magnetic Resonance (¹H NMR)

1D and 2D ¹H-NMR experiments were acquired on 600 MHz NMR spectrometer Bruker Avance III HD equipped with triple resonance cryoprobe at a temperature of 300K using DMSO-d6.

### Microanalysis (Elemental Analysis)

Microanalysis was performed using an Elemental Analyzer FLASH 2000 (Thermo Scientific) and using the acquisition and reprocessing software Eager Xperience V1.3. Analysis were done according to the protocol EP_ANA_001_Analyse CHNS_V02.

### Liquid Chromatography Mass Spectrometry (LC-MS)

LC-MS samples were analysed at a concentration of 0.125 mg/mL in DMSO using a Waters Acquity UPLC and an Aquity UPLC C18 column (dimensions 2.1×50 mm, particle size 1.7 uM) at an oven temperature of 55° C. at a flow rate of 1 mL/min. The gradient employed was composed of 2 eluents: Eluent A, containing 0.02% v/v of formic acid in water; and eluent B composed of 0.02% v/v of formic acid in acetonitrile. The gradient ran from 0.2%-98% eluent B over 4 minutes, held at 98% eluent B for 4.5 minutes before reducing to 2% eluent at 3.6-5.0 minutes.

### Crystallization Conditions

Ninety-six salts/crystallization conditions were tried and this first round of screening yielded seven salt candidates based on crystallinity testing. Any salts exhibiting a lack of crystallinity (e.g. were amorphous or had unstable polymorphic states) were not pursued at this stage. Of the seven candidates, four were further eliminated based on drawbacks, for example the potential to react with the API itself or excipients in a formulation, propensity to form genotoxic species, or because of their potential for forming mixtures of salts and co-crystal forms. The three salts described herein were found to provide stable crystalline compounds.

### Example 1: Synthesis of Compound I

Compound I was synthesized following the procedure as provided in Example 1 of PCT/EP2018/086066 and as provided below in Scheme 1.

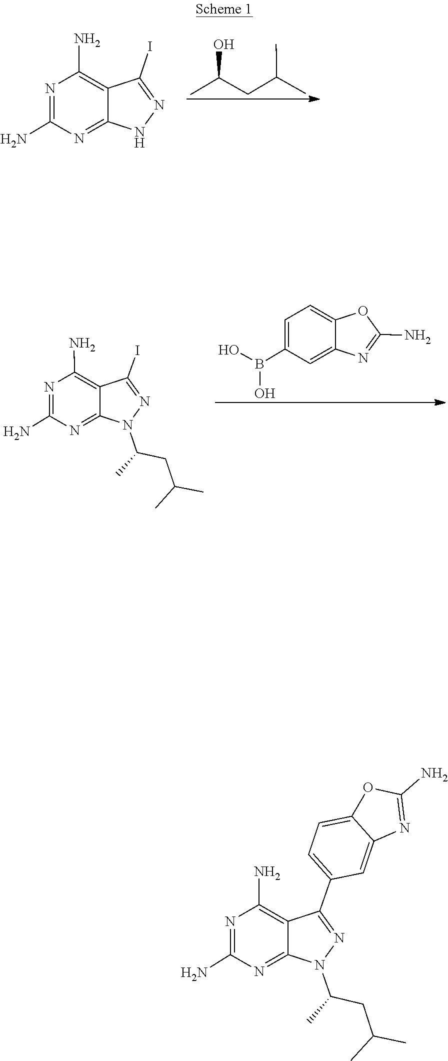

The route for synthesizing the compound 3-(2-aminobenzoxazol-5-yl)-1-((S)-1,3-dimethylbutyl)-1H-pyrazolo[3,4-d]pyrimidine-4,6-diamine is illustrated in Scheme 1.

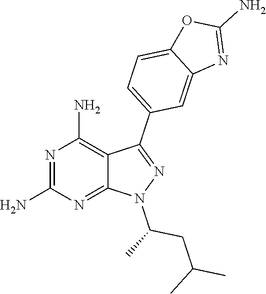 [SMILES: CC(C)C[C@H](C)n1nc(-c2ccc3oc(N)nc3c2)c2c(N)nc(N)nc21]

### Step 1: Synthesis of 1-((S)-1,3-dimethylbutyl)-3-iodo-1H-pyrazolo[3,4-d]pyrimidine-4,6-diamine

(R)-4-Methylpentan-2-ol (0.25 ml; 2.0 mmol; 1.1 eq.) is added to 3-iodo-1H-pyrazolo[3,4-d]pyrimidine-4,6-diamine (500 mg; 1.8 mmol; 1.0 eq.) (1) and triphenylphosphine (1.43 g; 5.4 mmol; 3.0 eq.) dissolved in tetrahydrofuran (10 ml). The reaction medium is cooled to 0° C. and diisopropyl azodicarboxylate (1.1 ml; 5.4 mmol; 3.0 eq.) is then added. The reaction medium is stirred at room temperature for 30 minutes. The reaction is stopped by adding water and cold iN sodium hydroxide solution to basic pH, and the mixture is then extracted with ethyl acetate. The organic phase is washed with saturated sodium chloride solution, dried over sodium sulfate and concentrated. The crude product is chromatographed on silica gel (25 g, solid deposition, dichloromethane/methanol eluent from 0 to 15% of methanol). 1-((S)-1,3-Dimethylbutyl)-3-iodo-1H-pyrazolo[3,4-d]pyrimidine-4,6-diamine (308 mg; 47%) is obtained in the form of a pale yellow foam.

### Step 2: Synthesis of 3-(2-aminobenzoxazol-5-yl)-1-((S)-1,3-dimethylbutyl)-1H-pyrazolo[3,4-d]pyrimidine-4,6-diamine

A solution of 1-((S)-1,3-dimethylbutyl)-3-iodo-1H-pyrazolo[3,4-d]pyrimidine-4,6-diamine (570 mg; 1.6 mmol; 1.0 eq.), (2-aminobenzo[d]oxazol-5-yl)boronic acid dihydrochloride (407 mg; 1.9 mmol; 1.2 eq.) and a solution of potassium carbonate (2.4 ml; 2.0 M; 4.8 mmol; 3.0 eq.) in 1,4-dioxane (5.7 ml) is degassed under nitrogen for 10 minutes, and 1,1′-bis(diphenylphosphino)ferrocenepalladium(II) dichloride, dichloromethane (65 mg; 0.08 mmol; 0.05 eq.) is then added. The medium is heated at 110° C. for 30 minutes. The reaction is stopped by adding water and the mixture is then extracted with ethyl acetate. The organic phases are combined, washed with saturated sodium chloride solution, dried over sodium sulfate and concentrated. The crude product is chromatographed on silica gel (12 g, solid deposition, dichloromethane/methanol eluent from 0 to 15% of methanol, TLC: dichloromethane/methanol 95/5 Rf=0.07). 3-(2-Aminobenzoxazol-5-yl)-1-((S)-1,3-dimethylbutyl)-1H-pyrazolo[3,4-d]pyrimidine-4,6-diamine (399 mg; 69%) is obtained in the form of a white crystalline solid after recrystallization from acetonitrile/water. 1H NMR (DMSO-d6) δ: 0.81 (d, J=6.5 Hz, 3H), 0.89 (d, J=6.5 Hz, 3H), 1.20-1.34 (m, 1H), 1.37 (d, J=6.7 Hz, 3H), 1.52 (ddd, J=13.6, 8.6, 5.1 Hz, 1H), 2.00 (ddd, J=13.6, 10.0, 5.4 Hz, 1H), 4.72-4.82 (m, 1H), 6.11 (s, 4H), 7.19 (dd, J=8.2, 1.8 Hz, 1H), 7.36 (d, J=1.9 Hz, 1H), 7.44 (dd, J=8.1, 5.2 Hz, 1H), 7.50 (s, 2H). MS (ESI) m/z=367 [M+H]+.

Peaks from the XRPD pattern are provided in Table 1.

TABLE 1
XRPD Peak list of phosphate salt

| Peak Name | Angle (2θ) | Rel. Intensity (%) |
|---|---|---|
| #1 | 7.897° | 44.2 |
| #2 | 9.391° | 2.5 |
| #3 | 10.193° | 29.2 |
| #4 | 11.988º | 12.3 |
| #5 | 13.797° | 10.7 |
| #6 | 14.855° | 17.1 |
| #7 | 15.871° | 100 |
| #8 | 16.863° | 12.1 |
| #9 | 17.618° | 24.7 |
| #10 | 18.015° | 3.5 |
| #11 | 18.768° | 2.3 |
| #12 | 19.471° | 4.2 |
| #13 | 20.077° | 6.1 |
| #14 | 20.931° | 9 |
| #15 | 21.767° | 7 |
| #16 | 22.139° | 8.8 |
| #17 | 23.666° | 16.3 |
| #18 | 23.927° | 22.1 |
| #19 | 25.128° | 11 |
| #20 | 26.691° | 2.7 |
| #21 | 27.417° | 2.9 |
| #22 | 28.461° | 6.8 |
| #23 | 30.057° | 5 |

DSC analysis of the free base provided a fusion onset at 254.79° C.

Elemental Analysis of the free base: Anal. Calcd for C₁₈H₂₂N₈O. 0.02 C3H8O. 0.018 H2O: C, 58.95; H, 6.075; N, 30.45. Found: C, 58.08; H, 6.025; N, 30.42.

### Example 2: Preparation and Characterization of Phosphate Salt of Compound I

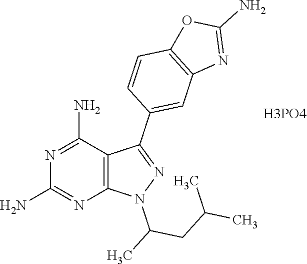

To a stirred suspension of 3-(2-amino-1,3-benzoxazol-5-yl)-1-(1,3-dimethylbutyl)pyrazolo[3,4-d]pyrimidine-4,6-diamine (1.50 g, 4.09 mmol) in ethanol (30 mL), was added at room temperature, a solution of phosphoric acid (401 mg, 4.09 mmol) in ethanol (8 mL). The reaction mixture turned homogeneous and then was heated up to 60° C. and stirred at 60° C. for 1 h. A suspension started to appear when the temperature reached 42° C. After 1 h at 60° C., heating was stopped, and the reaction mixture was slowly cooled down to room temperature and stirred overnight. The white heterogeneous reaction mixture was cooled to 10° C., stirred for 1 h and then filtered. The white solid was then dried in vacuo overnight to afford the phosphoric acid salt as a white solid. 1H NMR (DMSO-d6) δ: 0.84 (d, J=6.6 Hz, 3H), 0.89 (d, J=6.6 Hz, 3H), 1.27 (ddt, J=13.1, 8.5, 6.6, 6.6 Hz, 1H), 1.36 (d, J=6.6 Hz, 3H), 1.51 (ddd, J=13.7, 8.6, 5.1 Hz, 1H), 1.98 (ddd, J=13.7, 10.0, 5.4 Hz, 1H), 4.76 (ddd, J=10.0, 6.5, 5.3 Hz, 1H), 6.17 (br s, 4H), 7.19 (dd, J=8.1, 1.7 Hz, 1H), 7.35 (d, J=1.7 Hz, 1H), 7.43 (d, J=8.3 Hz, 1H), 7.51 (s, 2H), 8.59 (s, 2H).

The characteristic XRPD pattern is shown in FIG. 1. Peaks from the XRPD pattern are provided in Table 2.

TABLE 2
XRPD Peak list of phosphate salt

| Angle | d value | Intensity | Rel. Intensity |
|---|---|---|---|
| [° 2θ] | [Å] | [Count] | [%] |
| 2.836 | 31.12501 | 506 | 12.6 |
| 5.844 | 15.11012 | 4016 | 100.0 |
| 10.515 | 8.40659 | 203 | 5.1 |
| 11.630 | 7.60284 | 2010 | 50.0 |
| 16.147 | 5.48465 | 745 | 18.6 |
| 16.383 | 5.40616 | 770 | 19.2 |
| 17.687 | 5.01062 | 1707 | 42.5 |
| 18.738 | 4.73182 | 843 | 21.0 |
| 20.290 | 4.37330 | 1587 | 39.5 |
| 21.584 | 4.11387 | 1274 | 31.7 |
| 22.581 | 3.93438 | 1266 | 31.5 |
| 23.220 | 3.82759 | 1401 | 34.9 |
| 23.809 | 3.73426 | 1441 | 35.9 |
| 24.414 | 3.64301 | 1615 | 40.2 |
| 25.561 | 3.48213 | 1313 | 32.7 |
| 26.597 | 3.34877 | 1062 | 26.4 |
| 27.723 | 3.21528 | 826 | 20.6 |
| 28.512 | 3.12811 | 907 | 22.6 |
| 30.738 | 2.90638 | 622 | 15.5 |
| 32.631 | 2.74197 | 482 | 12.0 |
| 33.219 | 2.69478 | 527 | 13.1 |
| 36.153 | 2.48253 | 476 | 11.9 |
| 37.060 | 2.42384 | 477 | 11.9 |
| 37.582 | 2.39137 | 577 | 14.4 |

TGA analysis of the phosphate salt of Compound I provided a weight loss of 0.5% at 100° C. The TGA thermogram is provided in FIG. 2.

DSC analysis of the phosphate salt of Compound I provided a fusion onset at 208.88° C. The DSC thermogram is provided in FIG. 2.

The DSC thermogram recorded for the phosphate salt shows no significant thermal event before the large and narrow endothermic peak noted at ˜210° C. corresponding to the melting transition of the phosphate salt. However, the endothermic peak associated with the melting transition has been found concomitant to the thermal drug degradation noted by TGA. Due to this observation, melting parameters have only been roughly determined and reported for information. The TGA thermogram recorded for the phosphate salt shows a slight loss of mass of 0.5% between RT and 100° C. associated with the departure of volatile molecules and a large and continuous loss of mass above 200° C. associated with the thermal decomposition of the drug substance.

Elemental Analysis of the free base: Anal. Calcd for C18H22N8O. 0.129 H2O. 0.987 H3PO4: C, 46.44; H, 5.46; N, 24.07. Found: C, 45.44; H, 5.40; N, 23.61.

### Example 3: Preparation and Characterization of Malonic Acid Salt of Compound I

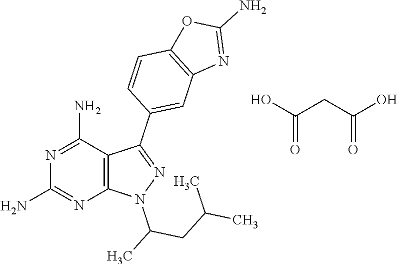 [SMILES: CC(C)CC(C)n1nc(-c2ccc3oc(N)nc3c2)c2c(N)nc(N)nc21 ; O=C(O)CC(=O)O]

To a stirred suspension of 3-(2-amino-1,3-benzoxazol-5-yl)-1-(1,3-dimethylbutyl)pyrazolo[3,4-d]pyrimidine-4,6-diamine (1.50 g, 4.09 mmol) in Methyl Ethyl ketone (20 mL), was added at 30° C., a solution of malonic acid (426 mg, 4.09 mmol) in Methyl Ethyl ketone (5 mL). The reaction mixture turned homogeneous and then was heated up to 60° C. and stirred at 60° C. for 1 h. A suspension started to appear when the temperature reached 47° C. After 1 h at 60° C., heating was stopped, and the reaction mixture was slowly cooled down to room over 2.5 h (26° C.). The white heterogeneous reaction mixture was then cooled to 10° C., stirred for 1 h and then filtered. The white solid was washed with Methyl Ethyl ketone (5 mL) then dried in vacuo overnight to afford the malonic acid salt as a white solid. 1H NMR (DMSO-d6) δ: 0.79 (d, J=6.6 Hz, 3H), 0.87 (d, J=6.6 Hz, 3H), 1.24 (ddt, J=13.1, 8.5, 6.6, 6.6 Hz, 1H), 1.36 (d, J=6.6 Hz, 3H), 1.51 (ddd, J=13.7, 8.6, 5.1 Hz, 1H), 1.99 (ddd, J=13.7, 10.0, 5.4 Hz, 1H), 3.21 (s, 2H), 4.76 (ddd, J=10.0, 6.5, 5.3 Hz, 1H), 6.21 (br s, 4H), 7.20 (dd, J=8.1, 1.7 Hz, 1H), 7.35 (d, J=1.7 Hz, 1H), 7.42 (d, J=8.3 Hz, 1H), 7.50 (s, 2H), 12.72 (br s, 1H).

The characteristic XRPD pattern is shown in FIG. 3. Peaks from the XRPD pattern are provided in Table 3.

TABLE 3
XRPD Peak list of malonic acid salt

| Angle | d value | Intensity | Rel. |
|---|---|---|---|
| [° 2θ] | [Å] | [Count] | Intensity [%] |
| 3.700 | 23.85841 | 295 | 5.0 |
| 4.853 | 18.19444 | 143 | 2.4 |
| 7.175 | 12.31122 | 652 | 11.0 |
| 7.529 | 11.73302 | 241 | 4.1 |
| 8.037 | 10.99140 | 410 | 6.9 |
| 8.691 | 10.16585 | 817 | 13.8 |
| 9.100 | 9.71008 | 315 | 5.3 |
| 10.704 | 8.25835 | 132 | 2.2 |
| 11.211 | 7.88628 | 144 | 2.4 |
| 12.325 | 7.17561 | 148 | 2.5 |
| 12.932 | 6.84012 | 785 | 13.3 |
| 13.338 | 6.63310 | 514 | 8.7 |
| 13.697 | 6.45984 | 308 | 5.2 |
| 14.373 | 6.15731 | 470 | 7.9 |
| 15.422 | 5.74102 | 333 | 5.6 |
| 15.833 | 5.59285 | 782 | 13.2 |
| 16.193 | 5.46938 | 1148 | 19.4 |
| 17.392 | 5.09477 | 630 | 10.7 |
| 18.221 | 4.86490 | 788 | 13.3 |
| 18.450 | 4.80505 | 897 | 15.2 |
| 19.546 | 4.53796 | 797 | 13.5 |
| 20.088 | 4.41681 | 1224 | 20.7 |
| 20.422 | 4.34531 | 709 | 12.0 |
| 21.227 | 4.18231 | 777 | 13.1 |
| 21.556 | 4.11920 | 697 | 11.8 |
| 22.172 | 4.00612 | 1016 | 17.2 |
| 23.172 | 3.83537 | 814 | 13.8 |
| 24.016 | 3.70257 | 5913 | 100.0 |
| 24.584 | 3.61830 | 2396 | 40.5 |
| 25.610 | 3.47552 | 1569 | 26.5 |
| 26.191 | 3.39972 | 2027 | 34.3 |
| 26.966 | 3.30381 | 716 | 12.1 |
| 27.677 | 3.22047 | 856 | 14.5 |
| 28.541 | 3.12493 | 1262 | 21.3 |
| 28.945 | 3.08224 | 806 | 13.6 |
| 30.226 | 2.95452 | 942 | 15.9 |
| 31.027 | 2.88004 | 516 | 8.7 |
| 31.569 | 2.83178 | 570 | 9.6 |
| 32.728 | 2.73409 | 609 | 10.3 |
| 33.635 | 2.66241 | 643 | 10.9 |
| 34.189 | 2.62053 | 541 | 9.1 |

TGA analysis of the malonic acid salt of Compound I provided a weight loss of 3.5% up to 140° C. The TGA thermogram is provided in FIG. 4.

DSC analysis of the malonic acid salt of Compound I provided fusion onset at 117.9° C.

The DSC thermogram recorded for the malonate salt shows no significant thermal events between RT and 100° C. and a large endothermic peak at ˜120° C. associated with the melting transition of the crystalline malonate salt. A shoulder on the low temperature wing of the melting transition is also noted. The TGA thermogram recorded for the malonate salt shows a loss of mass of ˜3.5% between RT and 130° C. associated with the departure of volatile molecules and a large loss of mass of ˜21.7% between 110° C. and 220° C. associated with the sublimation of malonic acid.

FT-IR Analysis of the malonic acid salt of Compound I is provided in FIG. 5.

Elemental Analysis of the free base: Anal. Calcd for C₁₈H₂₂N₈O. 0.03 C6H15N. 0.04 C3H60. 0.045 H2O: C, 53.67; H, 5.66; N, 23.60. Found: C, 53.80; H, 5.85; N, 22.50.

### Example 4: Preparation and Characterization of Malate Salt of Compound I

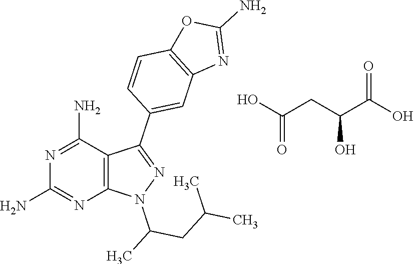 [SMILES: CC(C)CC(C)n1nc(-c2ccc3oc(N)nc3c2)c2c(N)nc(N)nc21 ; O=C(O)C[C@H](O)C(=O)O]

To a stirred suspension of 3-(2-amino-1,3-benzoxazol-5-yl)-1-(1,3-dimethylbutyl)pyrazolo[3,4-d]pyrimidine-4,6-diamine (1.50 g, 4.09 mmol) in acetonitrile (20 mL), was added at 30° C., a suspension of (S)-Malic acid (549 mg, 4.09 mmol) in acetonitrile (5 mL). The yellow heterogeneous reaction mixture was heated up to 60° C. and stirred at 60° C. for 3 h. After 3 h at 60° C., heating was stopped, and the reaction mixture was slowly cooled down to room temperature over 1.5 h. The white heterogeneous reaction mixture was then cooled to 10° C., stirred for 1 h and then filtered. The white solid was washed with acetonitrile (5 mL) then dried in vacuo overnight to afford the (S)-malic acid salt as a white solid. 1H NMR (DMSO-d6) δ: 0.81 (d, J=6.6 Hz, 3H), 0.89 (d, J=6.6 Hz, 3H), 1.24 (ddt, J=13.1, 8.5, 6.6, 6.6 Hz, 1H), 1.36 (d, J=6.6 Hz, 3H), 1.51 (ddd, J=13.7, 8.6, 5.1 Hz, 1H), 1.99 (ddd, J=13.7, 10.0, 5.4 Hz, 1H), 2.46 (m, 1H), 2.60 (m, 1H), 3.50 (m, 1H), 4.27 (m, 1H), 4.75 (ddd, J=10. 0, 6.5, 5.3 Hz, 1H), 6.15 (br s, 4H), 7.19 (dd, J=8.1, 1.7 Hz, 1H), 7.35 (d, J=1.7 Hz, 1H), 7.43 (d, J=8.3 Hz, 1H), 7.49 (s, 2H), 12.09 (br s, 11H).

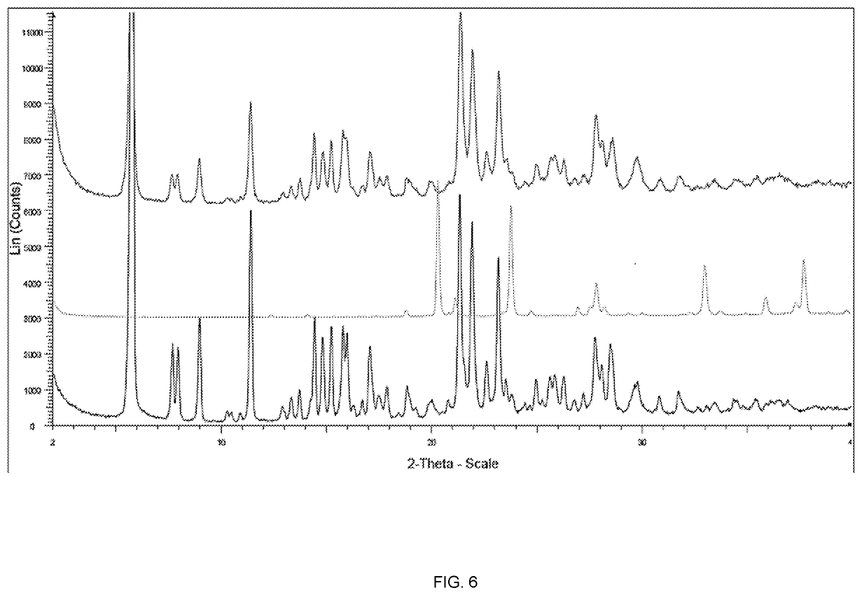

The characteristic XRPD pattern is shown in FIG. 6. Peaks from the XRPD pattern are provided in Table 4.

TABLE 4
XRPD Peak list of malate salt

| Angle | d value | Intensity | Rel. Intensity |
|---|---|---|---|
| [° 2θ] | [Å] | [Count] | [%] |
| 5.748 | 15.36174 | 28599 | 100.0 |
| 7.653 | 11.54227 | 1985 | 6.9 |
| 7.933 | 11.13528 | 1896 | 6.6 |
| 8.961 | 9.86032 | 2574 | 9.0 |
| 10.263 | 8.61211 | 419 | 1.5 |
| 10.438 | 8.46826 | 390 | 1.4 |
| 10.883 | 8.12270 | 367 | 1.3 |
| 11.402 | 7.75433 | 5078 | 17.8 |
| 12.915 | 6.84938 | 521 | 1.8 |
| 13.312 | 6.64562 | 717 | 2.5 |
| 13.723 | 6.44768 | 915 | 3.2 |
| 14.227 | 6.22043 | 688 | 2.4 |
| 14.417 | 6.13862 | 2606 | 9.1 |
| 14.811 | 5.97624 | 2134 | 7.5 |
| 15.236 | 5.81059 | 2375 | 8.3 |
| 15.776 | 5.61291 | 2394 | 8.4 |
| 15.941 | 5.55514 | 2220 | 7.8 |
| 16.268 | 5.44426 | 546 | 1.9 |
| 16.677 | 5.31179 | 676 | 2.4 |
| 17.061 | 5.19304 | 1915 | 6.7 |
| 17.478 | 5.07006 | 777 | 2.7 |
| 17.870 | 4.95966 | 978 | 3.4 |
| 18.818 | 4.71197 | 991 | 3.5 |
| 19.248 | 4.60760 | 501 | 1.8 |
| 19.863 | 4.46638 | 582 | 2.0 |
| 20.011 | 4.43350 | 686 | 2.4 |
| 20.772 | 4.27279 | 674 | 2.4 |
| 21.327 | 4.16293 | 5443 | 19.0 |
| 21.918 | 4.05200 | 4806 | 16.8 |
| 22.608 | 3.92975 | 1584 | 5.5 |
| 23.184 | 3.83342 | 3973 | 13.9 |
| 23.524 | 3.77883 | 1150 | 4.0 |
| 23.808 | 3.73438 | 806 | 2.8 |
| 24.428 | 3.64098 | 611 | 2.1 |
| 24.669 | 3.60602 | 549 | 1.9 |
| 24.972 | 3.56294 | 1153 | 4.0 |
| 25.251 | 3.52409 | 678 | 2.4 |
| 25.635 | 3.47222 | 1209 | 4.2 |
| 25.862 | 3.44225 | 1258 | 4.4 |
| 26.284 | 3.38798 | 1226 | 4.3 |
| 26.795 | 3.32442 | 660 | 2.3 |
| 27.227 | 3.27275 | 841 | 2.9 |
| 27.775 | 3.20939 | 2123 | 7.4 |
| 28.098 | 3.17320 | 1481 | 5.2 |
| 28.518 | 3.12741 | 1973 | 6.9 |
| 29.698 | 3.00577 | 1046 | 3.7 |

TGA analysis of the malate salt of Compound I provided a weight loss of 1.51% up to 350° C. The TGA thermogram is provided in FIG. 7.

DSC analysis of the malate salt of Compound I provided an endotherm at 186.6° C. The DSC thermogram is provided in FIG. 7.

The DSC thermogram recorded for the malate salt shows a weak and broad endothermic peak between RT and 90° C. (ie. in the temperature range where a loss of mass of ˜1.5% has been noted by TGA); and a large and narrow endothermic peak at ˜185° C. associated with the melting transition of the malate salt/Co-Crystal form. This melting transition is characterized by the following melting parameters: Melting Temperature: Tmelt=186.6° C.; and Melting enthalpy: ΔHmelt=˜83.3 J/g. The TGA thermogram recorded for the malate salt shows a weak loss of mass (˜1.5%) between RT and 100° C.; no significant loss of mass between 100° C. and 180° C.; and a large and continuous loss of mass above 180° C. associated with the thermal decomposition of the drug substance.

Elemental Analysis of the free base: Anal. Calcd for C₁₈H₂₂N₈O. C4H605. 0.452 H2O: C, 51.96; H, 5.73; N, 22.03. Found: C, 51.46; H, 5.58; N, 21.88.

### Example 5: Kinetic Solubility of Free Base, Phosphate Salt, Malonate Salt, and Malate Salt in Water, Water/Glycerin, Water/Transcutol and Glycerin at 37° C.

The following protocol has been used to prepare samples for solubility measurements at room temperature in the 6 different media. First, weigh appropriate amount of each salt and free base compound to reach the desired target concentration (30 mg/ml). Addition at room temperature the studied media to reach the selected target concentration, follow by orbital stirring at room temperature for 24 h, while protected from light. Except for the glycerin medium, separation of the soluble fraction from the non-soluble fraction is performed by centrifugation (15 min at 18000 rpm) followed by filtration on PTFE 0.45 μm membrane. For the glycerin medium, 3 steps of centrifugation (15 mw at 18000 rpm) to collect the supernatant was performed. The resulting filtrates and supernatant for glycerin medium were diluted in appropriate solvents described in the following table to be in the range of calibration curve. Quantification of the diluted and undiluted samples was performed using UPLC-UV. The dilution steps are prepared in triplicate according the following protocols, shown in Table 5:

TABLE 5
Dilution Protocol

| Media | Dilution Protocol | Dilution Media |
|---|---|---|
| H₂O | Free base undiluted | Mixture of water/ |
| | Malonate and malate | acetonitrile 50/50 (v/v) |
| | salts successive dilutions: | |
| | x10 and x100 | |
| | Phosphate salt successive | |
| | dilutions: x10; x100 and | |
| | x1000 | |
| Water/ | Free base undiluted | Mixture of medium/ |
| Glycerin 95/5 | 3 salts successive | acetonitrile 50/50 (v/v) |
| Water/ | dilutions: x10; x100 and | Mixture of medium/ |
| Glycerin 90/10 | x1000 | acetonitrile 50/50 (v/v) |
| Water/ | | Mixture of medium/ |
| Glycerin 80/20 | | methanol 50/50 (v/v) |
| Water/Transcutol | | Mixture of medium/ |
| 90/10 | | acetonitrile 50/50 (v/v) |
| Glycerin | Free base and 3 salts | Mixture of glycerin/ |
| | successive | methanol 50/50 (v/v) |
| | dilutions: x10; x100 | for dilution x 10 |
| | and x1000 | Mixture of glycerin/ |
| | | methanol 25/75 (v/v) |
| | | for dilution x100 |
| | | and x1000 |

A standard calibration curve (chromatographic UV peak area vs concentration) between 0.5 μg/mL and 500 μg/mL was established in DMSO according to the following protocol. First, weighing 1 mg free base, then addition of DMSO until a 1 mg/mL clear DMSO Stock solution is obtained. Next, selected compound DMSO solutions at 5 different concentrations (500 ag/mL, 250 ag/mL, 100 ag/ml, 25 μg/ml and 0.5 μg/mL) were prepared by dilution with pure DMSO.

Samples were analysed by UPLC-UV-MS according to the following: (a) Instrument: Waters Acquity H-Class with PDA and QDa detectors; (b) Column: Waters Acquity BEH C18, 1.7 m, 2.1×50 mm; (c) Flow rate: 0.65 ml/min; (d) UV detection: 220 nm, 254 nm, 290 nm; (e) Column temperature: 40° C.; (f) Injection volume: 0.4 μl; (g) Mobile phase: Gradient with solutions A and B prepared according to Table 6; (h) Ionisation mode: ESI+/ESI−; (i) Source temperature: 600° C.; (j) Capillary voltage: 0.8 kV; and (k) Cone voltage: +20V/−20V.

TABLE 6
UPLC gradient method used for solubility measurements

| Time | A = % Ultrapurified | B = % Acetonitrile + |
|---|---|---|
| in min. | water + 0.1% HCOOH | 0.05% HCOOH |
| 0 | 90 | 5 |
| 1.8 | 5 | 95 |
| 2.6 | 5 | 95 |
| 2.7 | 90 | 5 |
| 3.6 | 90 | 5 |

### Example 6: Solubility Measurements

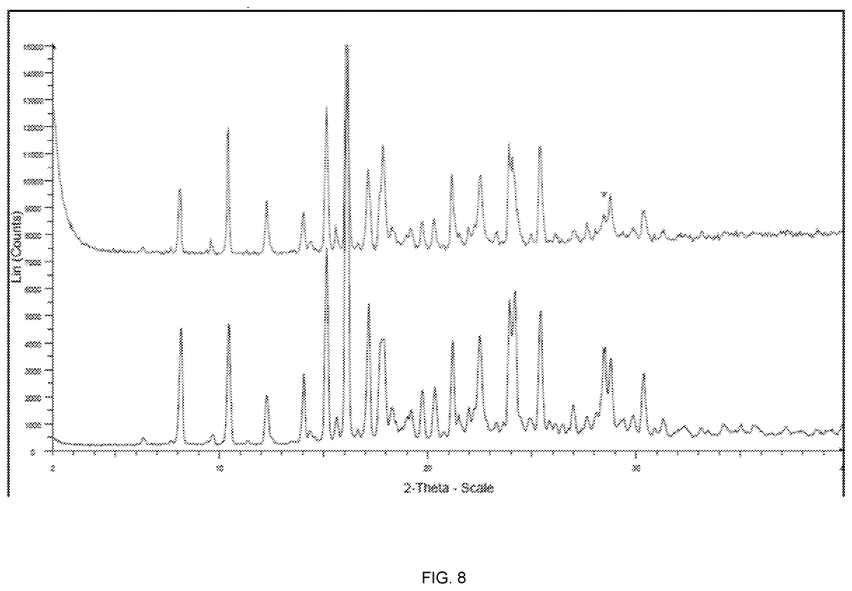

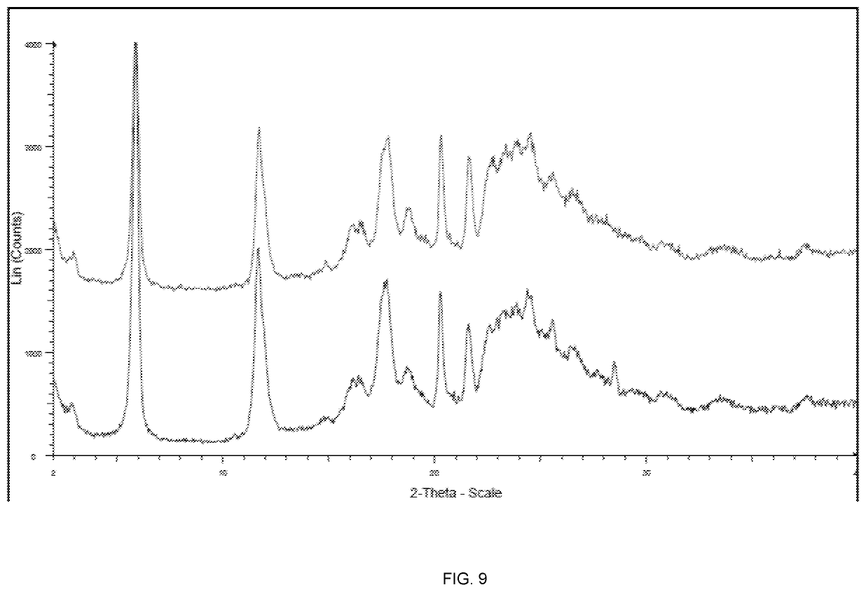

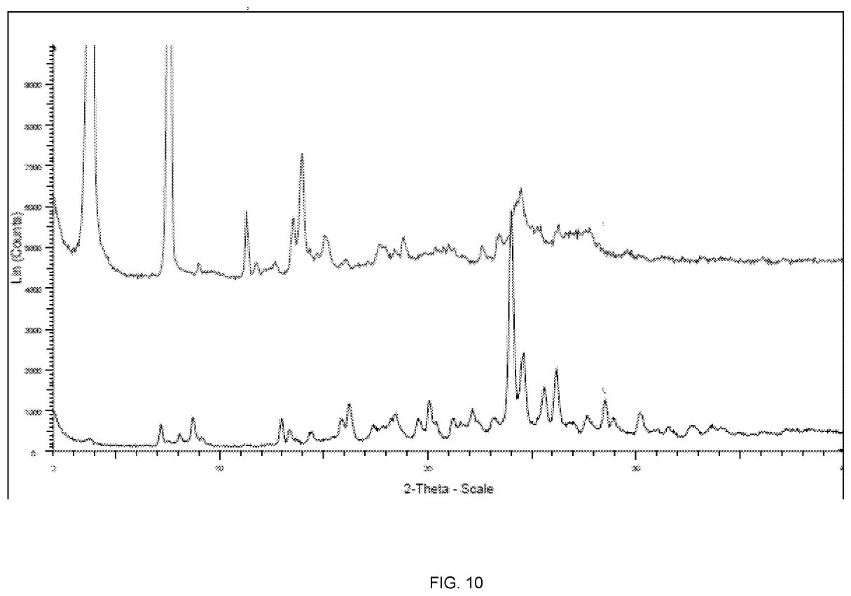

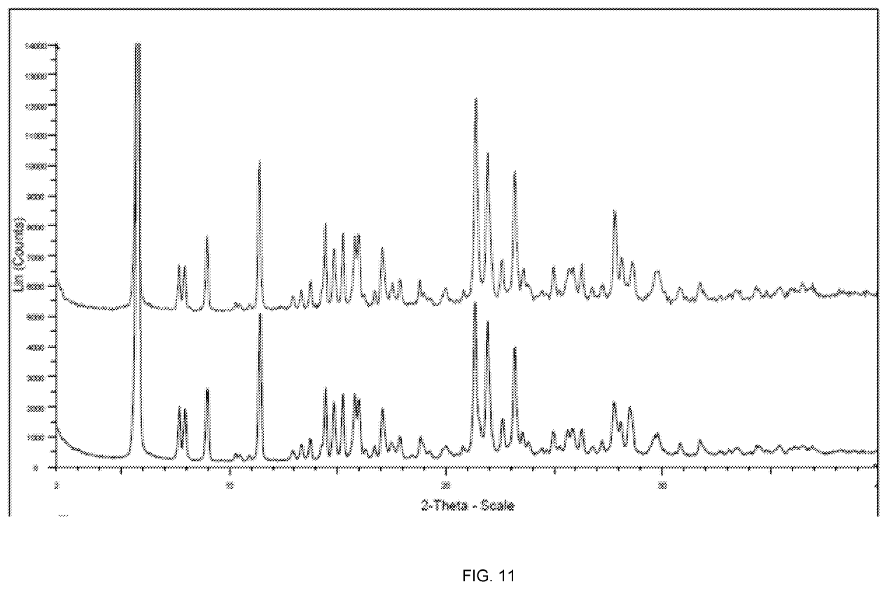

The target concentration tested to measure the maximum solubility of the 3 salts (phosphate, malonate and malate) and free base in 6 different media was 30 mg/ml. Solids in suspension in water solubility assays have been collected by centrifugation (15 min at 18000 rpm) after 24 h stirring for XRPD analysis. Recorded XRPD patterns have been compared to the one recorded for the salts or free base as received (see FIGS. 8-11).

Solubility results and XRPD analysis of solid residues in water are reported in Tables 7 and Table 8.

TABLE 7
Solubility values of the salts (phosphate, malonate, and malate) in 6 different media.

| | Solubility of free base and salts (mg/ml) | | | | | |
|---|---|---|---|---|---|---|
| | | Transcutol/ | Glycerin/ | Glycerin/ | Glycerin/ | |
| | | Water 10/90 | Water 5/95 | Water 10/90 | Water 20/80 | |
| | Water | (v/v) | (v/v) | (v/v) | (v/v) | Glycerin |
| Free Base | 0.18 | 0.256 | 0.029 | 0.045 | 0.071 | 6.4 ± 0.3 |
| Phosphate | 3.9 ± 0.2 | 5.5 ± 0.2 | 4.3 ± 0.2 | 4.7 ± 0.2 | 5.7 ± 0.2 | 15.8 ± 0.3 |
| Salt | | | | | | |
| Malonate | 2.2 ± 0.0 | 3.7 ± 0.2 | 2.7 ± 0.2 | 2.9 ± 0.2 | 3.5 ± 0.2 | 24.7 ± 0.3 |
| Salt | | | | | | |
| Malate Salt | 1.6 ± 0.0 | 3.1 ± 0.3 | 2.1 ± 0.3 | 2.2 ± 0.2 | 2.9 ± 0.2 | 21.1 ± 0.2 |

TABLE 8
XRPD Analysis of Solid Residues in water.

| | XRPD of solid residues in water |
|---|---|
| Free Base | No crystalline phase conversion |
| Phosphate Salt | No crystalline phase conversion |
| Malonate Salt | Crystalline Phase conversion |
| | (but signal not well defined) |
| Malate Salt | No crystalline phase conversion |

### Example 7: Comparison of In Vitro Permeability for Free Base and Phosphate Salt

In vitro permeation testing was performed using human abdominal skin from 3 donors and 2 samples per donor with a thickness of 400 μm (±100 μm). The samples were used in Franz cell 2 cm² of diameter and a receptor volume of 3 mL (PBS+4% BSA (Bovine Serum Albumin).

The receptor compartment was surrounded by a water jacket heated to 37° C.±1° C. insuring a skin surface temperature of 32° C.±1° C. The receptor compartment is separated from the donor compartment by the skin membrane where the epidermis side faces the donor. Receptor compartments containing magnetic stirrer bar, were filled with the receptor fluid in such a way to prevent any formation of air-bubbles. During the diffusion experiment, receptor fluids were continuously stirred in order to ensure homogenization.

Abdominal skins from aesthetic surgery were used in this study. Skins, upon arrival, were separated from hypodermis with the help of tweezers and a scalpel blade. Skins were gently washed and stored flat in aluminum foil for freezing at −20° C. At the day of experiment, skins were thawed then cut in smaller pieces to fit with diffusion cell geometry. In this experiment three donors were used. Skin specimens were mounted onto Franz diffusion cells with PBS+4% Bovine Serum Albumin as a receptor fluid. Average skin thickness was 0.4±0.1 mm. Thicknesses of all specimens are available in archives.

Following at least 45 min equilibrium with receptor fluid, skin integrity is assessed by Transepidermal Water Loss (TEWL) measurements. All cells where TEWL measurements exceed acceptance criteria (9 g/m²/h for abdomen or mammary skin) are carefully wiped off and allowed to equilibrate for an extended time. TEWL is again measured and cells still depicting a high water flux are recorded. Average TEWL, room temperature and relative humidity were all in the range of accepted values. Individual TEWL values for each skin specimen used, room temperature and relative humidity were also recorded.

Test Items were applied at a dose of 5 mg/cm² for 24 hours on the skin samples in the Franz cell. At the end of exposure, the following samples were recovered: Stratum corneum (not analysed), epidermis, dermis and receptor fluid were collected analysed for API content.

The samples were analyzed following LC/MS/MS method internally certified. The major differences in performance between the free base and salt at an equivalent concentration of active (0.5 w/w % of active free base and 0.63 w/w % of phosphate salt, which is equivalent to 0.5 w/w % of free base) were noted in terms of percutaneous flux and skin permeability coefficient (Kp). Despite replacing the 10% w/w of propylene glycol of the free base formulation with water, the formulation containing the phosphate salt shows superior permeability. Kp for the phosphate salt was 3 times higher than Kp for the free base. Even more striking, the ratio of percutaneous flux over dermis concentration was over 600 times greater for the phosphate salt compared to the free base. Percutaneous fluxes were calculated as the ratio of permeated amounts in receptor fluid (RF) of each individual cell to the duration of exposure (24 h).

### EQUIVALENTS

While certain embodiments have been illustrated and described, a person with ordinary skill in the art, after reading the foregoing specification, can effect changes, substitutions of equivalents and other types of alterations to the compounds of the present technology or salts, pharmaceutical compositions, derivatives, prodrugs, metabolites, tautomers or racemic mixtures thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, reagents, compounds, compositions, labeled compounds or biological systems, which can, of course, vary. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims, along with the full scope of equivalents to which such claims are entitled.

What is claimed is:

1. A salt of a compound of Formula I: 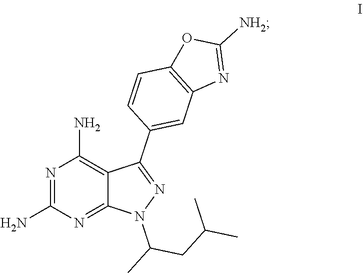 [SMILES: CC(C)CC(C)n1nc(-c2ccc3oc(N)nc3c2)c2c(N)nc(N)nc21]
wherein the salt is selected from phosphate, malonate, and malate.

2. The salt of claim 1, wherein the salt is crystalline.

3. A crystalline compound selected from a crystalline phosphate, malonate or malate salt of a compound of Formula I:

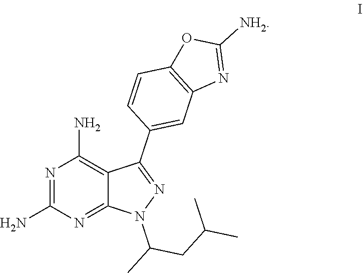 [SMILES: CC(C)CC(C)n1nc(-c2ccc3oc(N)nc3c2)c2c(N)nc(N)nc21]

4. The crystalline phosphate compound of claim 3, wherein the phosphate salt has an X-ray powder diffraction pattern comprising one or more characteristic peaks, in terms of 2θ, at about 5.8°, about 11.6°, and about 17.7°.

5. The crystalline phosphate compound of claim 4, wherein the X-ray powder diffraction pattern of the crystalline phosphate salt further comprises one or more characteristic peaks, in terms of 2θ, at about 24.4°, about 20.3°, and about 23.8°.

6. The crystalline phosphate compound of claim 4, wherein the X-ray powder diffraction pattern of the crystalline phosphate salt further comprises one or more characteristic peaks, in terms of 2θ, at about 23.2°, about 21.6°, about 22.6°, and about 26.6°.

7. The crystalline phosphate compound of claim 3 having:
(a) an X-ray powder diffraction pattern substantially as shown in FIG. 1; or
(b) a differential scanning calorimetry thermogram showing an onset at about 208.9° C.;
or
(c) a differential scanning calorimetry thermogram substantially as shown in FIG. 2.

8. The crystalline malonate compound of claim 3, wherein the malonate salt has an X-ray powder diffraction pattern comprising one or more characteristic peaks, in terms of 2θ, at about 24.0°, about 24.6°, and about 26.2°.

9. The crystalline malonate compound of claim 8, wherein the X-ray powder diffraction pattern of the crystalline malonate salt further comprises one or more characteristic peaks, in terms of 2θ, at about 25.6°, about 20.1°, and about 16.2°.

10. The crystalline malonate compound of claim 8, wherein the X-ray powder diffraction pattern of the crystalline malonate salt further comprises one or more characteristic peaks, in terms of 2θ, at about 22.2°, about 30.2°, about 18.5°, about 8.7°, and about 23.2°.

11. The crystalline form of claim 3 having:
(a) an X-ray powder diffraction pattern substantially as shown in FIG. 3; or
(b) a differential scanning calorimetry thermogram showing an onset at about 117.9° C.;
or
(c) a differential scanning calorimetry thermogram substantially as shown in FIG. 4.

12. The crystalline malate compound of claim 3, wherein the X-ray powder diffraction pattern comprises one or more characteristic peaks, in terms of 2θ, at about 5.7°, about 21.3°, and about 11.4°.

13. The crystalline malate compound of claim 12, wherein the X-ray powder diffraction pattern of the crystalline malate salt further comprises one or more characteristic peaks, in terms of 2θ, at about 21.9°, about 23.2°, about 14.4°, and about 8.9°.

14. The crystalline form of claim 3 having:
(a) an X-ray powder diffraction pattern substantially as shown in FIG. 6; or
(b) a differential scanning calorimetry thermogram showing an onset at about 186.6° C.;
or
(c) a differential scanning calorimetry thermogram substantially as shown in FIG. 7.

15. A pharmaceutical composition comprising an effective amount of the compound of claim 1 for treating an mTOR-mediated disorder or condition.

16. The pharmaceutical composition of claim 15, wherein the disorder or condition is a dermatological complaint associated with a keratinization disorder with a proliferative, inflammatory and/or immunoallergic component.

17. The pharmaceutical composition of claim 16, wherein the dermatological complaint is psoriasis, atopic dermatitis, actinic keratosis or acne.

18. A method of treatment comprising administering an effective amount of a compound of claim 1 to a subject suffering from an mTOR-mediated disorder or condition.

19. The method of claim 18, wherein the disorder or condition disorder or condition is a dermatological complaint associated with a keratinization disorder with a proliferative, inflammatory and/or immunoallergic component.

20. The method of claim 19, wherein the wherein the dermatological complaint is psoriasis, atopic dermatitis, actinic keratosis or acne.

21. A method comprising modulating mTOR by contacting mTOR with an effective amount of a compound of claim 1.
